(19) United States
(12) Patent Application Publication
(10) Pub. No.: US 2005/0029363 A1
(43) Pub. Date: Feb. 10, 2005

(54) Scent dispenser

(76) Inventor: William Brown, Opelika, AL (US)

Correspondence Address: MYERS & KAPLAN, INTELLECTUAL PROPERTY LAW, L.L.C., 1899 POWERS FERRY ROAD, SUITE 310, ATLANTA, GA, 30339, US

(21) Appl. No.: 10/634,142

(22) Filed: Aug. 4, 2003

Related U.S. Application Data
(63) Continuation-in-part of application No. 10/078,837, filed on Feb. 21, 2002, now abandoned.
(60) Provisional application No. 60/330,279, filed on Oct. 18, 2001.

(51) Int. Cl. A61L 9/04

(52) U.S. Cl. 239/55

(57) ABSTRACT

Provided is a scent dispenser having two tubular, slidably engageable members. Openings in the inner tubular member allow scent to be released into the surrounding environment when the tubular members are in an extended position. An absorbent material may be contained within the dispenser to carry a preferred scent. Cooperating, nestable retaining detents, or, alternatively, a selectively removably positionable elastomeric O-ring, allow the outer tubular member of the dispenser to be held in any of a plurality of extended positions. A cord may be attached to the top of the outer tubular member to allow the user to hang the dispenser in a desired location. The scent dispenser may be provided in a bright color to allow the dispenser further to function as a highly visible marker for measuring the effective range of a hunter's weapons.

### CROSS-REFERENCE AND PRIORITY CLAIM TO RELATED APPLICATIONS

To the full extent permitted by law, the present application claims priority to and the benefit of the following applications: 1) as a continuation-in-part application to non-provisional patent application entitled “Scent Dispenser,” filed on Feb. 21, 2002, and having assigned Ser. No. 10/078,837 (now abandoned), which claims priority to and the benefit of provisional patent application entitled “Scent Dispenser,” filed on Oct. 18, 2001, and having assigned Ser. No. 60/330,279.

### TECHNICAL FIELD

The present invention relates generally to scent dispenser devices for use in hunting and fishing activities; and, more particularly, to scent dispensers which are slidably adjustable, have a perforated area to allow the scent to disperse into the atmosphere, and are sealable.

### BACKGROUND OF THE INVENTION

Outdoorsmen have long used various baits to attract animals while hunting and fishing. A deer hunter, for example, may sprinkle estrous doe scent in areas upwind of his deer stand in order to attract bucks toward the stand. Estrous doe scent, however, can be very expensive. Accordingly, hunters have developed various devices and techniques for using such scents sparingly.

Similarly, a fisherman may find it advantageous to use chum for bait within a holding device, in order to attract fish into a fishing hole.

Equivalent requirements may be found with reference to law enforcement and human search-and-rescue operations, where containers of drug scent, gunpowder scent, or human scent may be used to train dogs to find contraband or to locate lost individuals.

It is clear that a suitable scent dispenser that may be resealed and reused would prove beneficial to a variety of users having differing requirements. A review of the prior art demonstrates that such devices have been developed and that they often fall into one of two principal categories.

Scent dispensers in the first category have both a large and a small member which slide together. One of the members has holes which are exposed when the two members are slid apart. Scented material is contained in the inner member and diffuses into the atmosphere when the holes are exposed.

Scent dispensers in the second category have a large tube with holes and a small tube with holes. The small tube slides into the large one. Scent bearing material is loaded into the small tube and the tubes are then sealed on both ends. When the two tubes rotate relative to one another the holes in both tubes line up and allow the scent to diffuse into the atmosphere.

With regard to the first type of dispenser:

U.S. Pat. No. 2,556,608 to Will discloses a multivent dispenser which is designed to be used in conjunction with an air conditioner to freshen a room. A bottle of liquid is housed in a lower section. An upper section with a plurality of holes fits over the lower section. A wicking mechanism allows the liquid to diffuse from the bottle, through the holes, and into the atmosphere. A detent mechanism allows the user to position the top portion at a desired height.

U.S. Pat. No. 1,481,325 to Le Gris discloses a moistener for cigars and tobacco. A partially perforated casing slides into another larger casing. A spring forces the casings into an open position. Absorbent material is held in the casing and is wetted with water. The water evaporates through the perforations to keep the tobacco moist. The moistener is placed in a cigar box. The sides of the box keep the casings from separating completely.

U.S. Pat. No. 2,763,395 to Meek discloses a diffuser device which comprises two cylindrical tubes. The smaller tube is perforated and expands to expose an array of holes. A scented material is contained within the small tube. Spring fingers engage when the small tube is slid out to keep it from retracting.

With regard to the second type of dispenser:

U.S. Pat. No. 2,438,129 to Rich discloses a vapor disseminator. The disseminator comprises a cylindrical container with holes and a cylindrical sleeve with holes. The two parts rotate relative to each other, allowing the holes in the container to line up with the holes in the sleeve. Scented material is contained in the cylindrical container and both ends are sealed. When the two parts are rotated, the holes align and allow the scent to disperse into the surrounding atmosphere.

U.S. Pat. No. 2,578,827 to Munnecke provides a small cylinder with holes to fit inside a large cylinder with holes. The scented material is absorbed by cotton or other absorbent material and is held in a foraminous shell inside the cavity of the small cylinder. The ends are plugged. The two cylinders are rotated relative to each other until they line up; thereby, allowing the scent to disperse into the surrounding atmosphere. The shell has enlarged areas to provide a snug fit in the small cylinder while the smaller areas provide for circulation of the scent.

In the prior art devices, the tendency has been to maximize the surface area of the openings, so as to maximize the flow of air through the device. This has resulted in devices which tend to over-saturate the air with scent. Over-saturating the air wastes the scented material, which is usually very expensive. Oversaturated air is an unnatural condition and may scare-off the very animals that the hunter wishes to attract.

Keeping the surface area of the openings small allows a more carefully metered and optimal amount of scent to be released into the air. This helps to keep the air from becoming oversaturated with scent.

None of the prior art devices disclose a device which allow the user to limit the scent dispersed to a minimal amount.

Still other scent dispensers, have been designed to preclude escape of the scented material or liquid via the incorporation of gaskets or other forms of seals, generally integrally formed therewith and/or retained within a channel or annular groove formed on one of the engaging members of the scent dispenser. Examples of such devices may be seen with reference to U.S. Pat. No. 2,738,224 to Turner et al.; U.S. Pat. No. 2,738,225 to Meek; U.S. Pat. No. 4,969,599 to Campbell; U.S. Pat. No. 5,388,762 to Bryson, Sr.; and, U.S. Pat. No. 6,102,301 to Tiedemann. Although the devices of the afore-referenced patents may incorporate a gasket or sealing ring for creating a spill-proof device, none appear to expand the functionality of the gasket or sealing ring; that is, the gasket or sealing ring is specifically limited to its sealing function. None of the devices of the afore-referenced patents appear to utilize the frictional qualities and/or removable positioning capabilities of the gasket or sealing ring to effectuate a wholly different and advantageous function. More specifically, absent from the prior art is user-selectable removably positionable sealing ring moveable from a first position to at least a second position, wherein the first position provides for a sealing function, thereby preventing spillage or loss of the scented material, and wherein the second position is user-selectable and provides a stop means for maintaining a first part or engaging member of the scent dispenser at a user-selected position or distance from a second part of the scent dispenser.

It is readily apparent that an improved scent dispenser is needed which is slidable between a fully closed position and an optimal operating position, limited only by contact being maintained between the two members. Preferably, the dispenser should provide a plurality of in-line openings, such as holes, through a side thereof, wherein at least one of the openings are located proximate the base of the dispenser, and wherein the extent of exposure of the openings may be adjusted by frictionally engageable, nestable detent retaining means upon each housing portion. Such a configuration would allow the user to adjust the dispenser to disperse an optimally minimal amount of scent. Additionally, the device may optionally incorporate a user-selectable removably positionable sealing ring for providing a sealing function or, alternatively, a stop means, wherein the stop means provides for adjusting the dispenser to a user-selected position for user-selected control of scent dispersion. It is, therefore, to the provision of such an improved scent dispenser that the present invention is directed.

### BRIEF SUMMARY OF THE INVENTION

The present invention is directed to an improved scent dispenser made of two preformed tubular members, preferably of rectangular cross-section. The first tubular member comprises an inner or male member, and the second comprises an outer or female member. The outer tubular member is disposed to slidably engage the inner tubular member in an overlying relationship. The engagement between the inner and outer member is infinitely adjustable between a fully-closed position and a fully-extended position, limited only by contact being maintained between the two members. When the tubular members are engaged, a cavity is formed which is disposed to contain a scent-bearing material.

In order to maintain a selected position, cooperating frictionally engageable, nestable detent retaining means, preferably disposed in a major face of the outer tubular member, frictionally engage similarly structured frictionally engageable detent retaining means in a corresponding major face of the inner tubular member. A plurality of frictionally engageable, nestable detent retaining means in the inner tubular member allow the outer tubular member to be affirmatively held in any of a plurality of pre-selected positions.

In order to carry the scent, cotton, cloth, or other absorbent material optionally may be placed within the cavity of the dispenser. Other scent carriers may be provided as disclosed hereinbelow.

In order to dispense the scent, the inner tubular member is perforated preferably with a plurality of opposed, in-line openings located in the minor faces of the inner tubular member. Preferably, the openings are located approximately 90 degrees from the frictionally engageable, nestable detent retaining means in the major faces.

In a round configuration, the openings are preferably rotated 90 degrees from the frictionally engageable, nestable detent retaining means, but could be located anywhere on the device, so long as such does not interfere with the openings. A means for positively aligning the nestable detent retaining means is desirable when using a round configuration. These alignment means may include a key and slot arrangement, or such other configurations as are well-known in the art.

In an alternate configuration, an exteriorly positioned elastomeric O-ring, disposed proximal the base of the inner tubular member, is incorporated for purposes of creating a spill-proof scent dispenser, wherein a flange formed around the base of the outer tubular member is dimensioned to engage the O-ring when the scent dispenser is in a fully-closed position, thereby preventing spillage of the scented material and/or fluid upon transport of the scent dispenser. Additionally, the elastomeric O-ring is selectively removably positionable along the length of the inner tubular member, thereby providing an adjustable stop means for frictional engagement with the flange of the outer tubular member; thus maintaining outer tubular member at a user-selected position for selectively controlling scent dispersion.

In all embodiments of the present invention, the inner tubular member is slid from within the outer tubular member a required distance in order expose a desired number of openings. The scent is then free to disperse into the surrounding atmosphere. The relatively long distance between minor faces and the relatively small hole size allows the user to adjust the scent dispenser so that only a small amount of scent may be dispersed.

In order to removably mount or carry the dispenser, a cord or strap is affixed to the outer tubular member by means well known in the art, and is used to position the dispenser in its desired location.

The dispenser can be formed in a variety of shapes and colors. Natural colors, such as green, brown, or tan, may be used to allow the dispenser to blend into trees or foliage. Alternatively, the use of bright colors, such as red, orange, or yellow, will allow the dispenser to also function as a highly visible marker useful for indicating the effective range of a hunter's weapons.

The dispenser of the present invention has a number and variety of uses, including, but not limited to: dispensing deer scent for hunting, holding chum for fish bait, dispensing fragrance for the car or home, dispensing insect repellent, and dispensing an animal, human, drug, or explosive scent for training dogs. Other uses are limited only by the imagination and requirements of the user.

Accordingly, a feature and advantage of the present invention is its ability to provide a scent dispenser which is infinitely variable between a fully closed position and a fully open position, limited only by contact being maintained between the two members.

Another feature and advantage of the present invention is its ability provide a scent dispenser which has frictionally engageable, nestable detent retaining means to retain the dispenser portions in the desired relative position.

Still another feature and advantage of the present invention is its ability to provide a scent dispenser which has openings, such as holes, in at least one side, and cooperating adjustment means, such as nestable detent means, in at least one other side to allow the scent to disperse into the atmosphere.

Yet another feature and advantage of the present invention is its ability to provide a scent dispenser that has a cord or, alternatively, a strap proximate to the top for permitting the dispenser to be conveniently carried or hung upon a tree.

Yet still another feature and advantage of the present invention is its ability to provide a scent dispenser which can be closed completely in order to keep the scent from diffusing into the atmosphere.

A further feature and advantage of the present invention is its ability to provide a scent dispenser which may be naturally colored in order to allow it to blend into the natural environment.

Still a further feature and advantage of the present invention is its ability to provide a brightly colored scent dispenser which may also be used to mark the effective range of a hunter's weapons.

Yet a further feature and advantage of the present invention is its ability to provide a scent dispenser which allows the user to better control the amount of scent dispersed.

Yet still a further feature and advantage of the present invention is its ability to provide a scent dispenser which allows the user to disperse an effectively minimal amount of scent.

Yet still another feature and advantage of the present invention is its incorporation of an elastomeric O-ring for purposes of creating a spill-proof scent dispenser.

Yet still another feature and advantage of the present invention is its incorporation of an elastomeric O-ring capable of being selectively removably positioned along the length of the inner tubular member for providing an adjustable stop means to maintain the outer tubular member at a user-selected position for selective control of scent dispersion.

These and other features and advantages of the present invention will become more apparent to one skilled in the art from the following description and claims when read in light of the accompanying drawings.

### BRIEF DESCRIPTION OF THE DRAWINGS

The present invention will be better understood by reading the Detailed Description of the Preferred and Alternate Embodiments with reference to the accompanying drawing figures, in which like reference numerals denote similar structures and refer to like elements throughout, and in which:

FIG. 1 is a perspective view of the preferred embodiment of the scent dispenser of the present invention, demonstrating the two tubular members in as-assembled condition, along with openings, nestable detent retaining means, and cord;

FIG. 2 is a partial sectional view of the preferred embodiment of the scent dispenser of the present invention, showing the two tubular members in as-assembled condition, along with openings, nestable detent retaining means, cavity, and scent bearing material;

FIG. 3 is a partial sectional view of a scent dispenser according to an alternate embodiment of the present invention;

FIG. 4 is a partial sectional view of a scent dispenser according to an alternate embodiment of the present invention;

FIG. 5 is a partial sectional view of a scent dispenser according to an alternate embodiment of the present invention;

FIG. 6A is a partial sectional view of a scent dispenser according to an alternate embodiment of the present invention;

FIG. 6B is a partial sectional view of a scent dispenser according to an alternate embodiment of the present invention;

FIG. 6C is a partial sectional view of a scent dispenser according to an alternate embodiment of the present invention; and,

FIG. 6D is a partial sectional view of a scent dispenser according to an alternate embodiment of the present invention.

### DETAILED DESCRIPTION OF THE PREFERRED AND ALTERNATE EMBODIMENTS

In describing preferred embodiments of the present invention illustrated in the Figures, specific terminology is employed for the sake of clarity. The invention, however, is not intended to be limited to the specific terminology so selected, and it is to be understood that each specific element includes all technical equivalents which operate in a similar manner to accomplish a similar purpose.

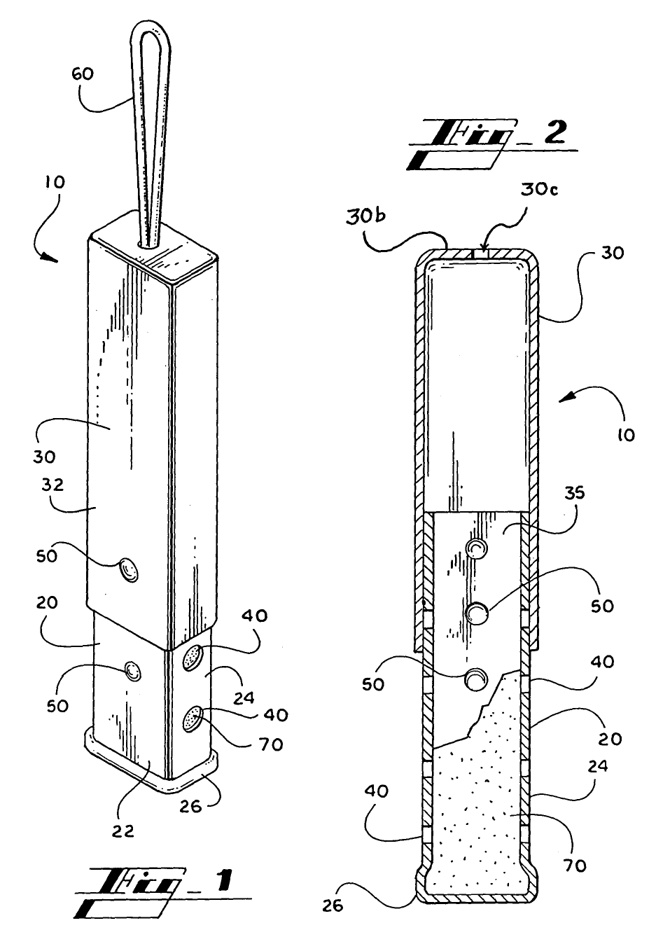

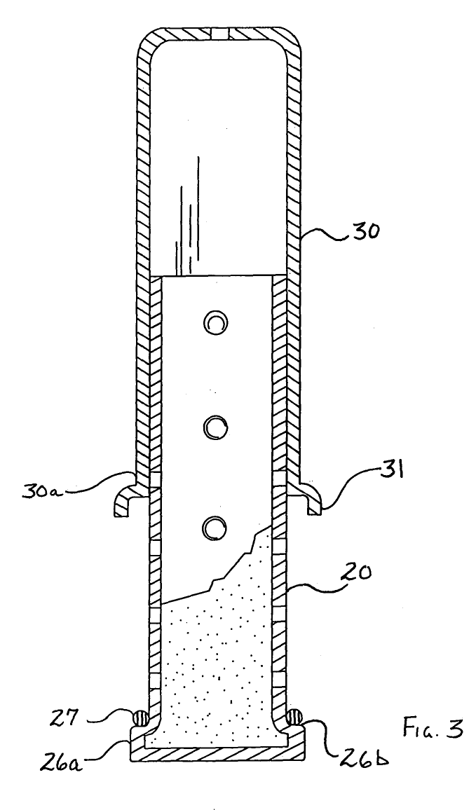

In accordance with the several objects of the present invention, and with reference to FIGS. 1 and 2, provided is scent dispenser 10 which is made of two preformed tubular members 20, 30, preferably of thermoformed plastic material. First tubular member 20 comprises an inner or male member. Second tubular member 30 comprises an outer or female member. Outer tubular member 30 is disposed to slidably engage inner tubular member 20 in an overlying relationship, infinitely variable between a fully-closed position and a fully-extended position, limited only by contact being maintained between the two members.

It will be apparent to one of ordinary skill in the art that tubular members 20, 30 may be rectangular, square, round, or any other equivalently functional shape.

The bottom of inner tubular member 20 is enlarged to provide base 26 for standing device 10 in a vertical position, to make device 10 easy to grasp, and to provide a stop to prevent inner tubular member 20 from being inserted too far into outer tubular member 30.

When tubular members 20, 30 are engaged, cavity 35 is formed, in which may be contained and carried scent bearing material 70. Scent bearing material 70 may be an absorbent material such as cloth or cotton. It may also be desirable to hold certain scent bearing materials directly within cavity 35, without the use of any absorbent material. Such scent bearing material may include deer scent for hunting; holding chum for fish bait; fragrance for car or home; insect repellent; or, animal, human, drug, or explosive scents for training dogs. Other scents and uses are limited only by the imagination and requirements of the user.

In order to dispense the scent, inner tubular member 20 carries openings 40 in minor face 24 and in its opposing symmetrical face. Preferably, a single line of spaced-apart openings 40 disposed centrally along one minor face 24 are arranged in paired, aligned relationship with equivalent openings 40 in the opposing face 24. Such arrangement allows air to flow into one side of device 10 and out of the other side. This results in a steady and more consistent scent dispersal, even when a minimum number of openings 40 are uncovered.

Openings 40 may be covered over by outer tubular member 30 when inner tubular member 20 is advanced fully into outer tubular member 30. As inner tubular member 20 is withdrawn from outer tubular member 30, an increasing number of openings 40 are exposed. The relatively long distance between minor faces 24 and the relatively small size of openings 40 allows the user to adjust scent dispenser 10 so that only a small amount of scent is dispersed, if so desired.

In order to maintain a desired closed or extended position, frictionally engageable, nestable detent retaining means 50 in major face 22 of inner member 20 and its opposing symmetrical face are disposed to frictionally engage, in nested arrangement, retaining means 50 in major face 32 of outer member 30 and its opposing symmetrical face. In the preferred embodiment shown, frictionally engageable, nestable detent retaining means 50 are best described as approximately hemispherical “dimples” formed in the described surfaces of members 20, 30. Such an arrangement allows dispenser 10 to be held within a range of positions which are preferred by the user.

It is noted that detent retaining means 50, when engaged in cooperating nested fashion, provide for affirmatively holding members 20, 30 in any of a plurality of pre-selected positions. Such positions preferably are pre-selected in order to allow full exposure of a next consecutive opening 40 along minor faces 24.

Notwithstanding, it is preferred that detent retaining means 50 in tubular member 30 be sufficiently rigid to provide sufficient friction in bearing against tubular member 20 so as to maintain members 20, 30 in any of a plurality of positions between a fully-closed and a fully-extended position. Such an arrangement allows partial exposure of a next consecutive opening 40 along minor faces 24.

In a rectangular configuration, faces 22, 24 are located 90 degrees apart. In a round configuration, however, frictionally engageable, nestable detent retaining means 50 and openings 40 are preferably located 90 degrees apart. Although not essential to the function of the present invention, such configuration precludes interference of detent retaining means 50 with openings 40.

Accordingly, in a rectangular configuration, the shape of inner tubular member 20 and outer tubular member 30 hold these two members in the preferred position relative to each other. When other shapes are chosen, however, it may be desirable to include a locating means to orient the two members in the proper position relative to each other. Such means might include a key-and-slot arrangement, or such other configurations as are well-known in the art. The locating means may be placed in any convenient position on inner tubular member 20 and outer tubular member 30, so long as such means do not interfere with openings 40 or frictionally engageable detent retaining means 50.

In order to removably mount or carry scent dispenser 10, cord 60, strap, or other attaching means, well-known in the art, may be provided in association with outer tubular member 30. Cord 60 may be used to hang dispenser 10 in a desired location. Preferably, cord 60 extends from throughhole 30c centrally formed through top surface 30b of outer tubular member 30, wherein cord 60 is preferably secured therewithin via tying or knotting the end of cord 60; however, other suitable fasteners could be utilized, such as, for exemplary purposes only, rivets, epoxies, resins, hook-and-loop fasteners, or the like.

Dispenser 10 may be formed in a variety of shapes and colors. Natural colors, such as green, brown, or tan, may be used to allow dispenser 10 to blend into trees or foliage. Alternatively, the use of bright colors, such as red, yellow, or bright orange, may allow the dispenser to function also as a highly visible marker for measuring the effective range of a hunter's weapons.

Dispenser 10 may be used in a number of ways including: dispensing deer scent for hunting, holding chum for fish bait, dispensing fragrance for the car or home, dispensing insect repellent, and dispensing an animal, human, drug, or explosive scent for training dogs, all of which scents may be held within cavity 35.

In operation, the user slips inner tubular member 20 and outer tubular member 30 apart, and fills cavity 35 with a scented material 70 of his choice. Inner tubular member 20 and outer tubular member 30 are then slid together fully so that dispenser 10 can be conveniently transported without excessive loss of scent. When the user chooses to use dispenser 10, he slides tubular members 20, 30 apart in order to expose the desired number of openings 40, and hangs or otherwise mounts it at the location of his choice. The scent is then free to circulate into the air of the surrounding area. Advantageously, use of the scent dispenser of the present invention allows the user to vary and control the quantity of scent delivered into the environment, without loss, and without unnatural saturation of that environment.

In view of the above-described exemplary embodiments, it will be apparent to one ordinarily skilled in the art that variously shaped scent dispenser housings may be provided without departing from the scope of the present invention. For example, tubular members 20, 30 could have a rectangular, square, round, or irregularly shaped cross-section. Openings 40 could be of different sizes, shapes, and arrangement. Base 26 and frictionally engageable, nestable detent retaining means 50 could be provided in different geometric configurations. Cord 60 could be made of any of a variety of different materials, or could be substituted for other well-known positioning means, such as hook-and-loop fasteners, adhesives, or the like. Frictionally engageable, nestable detent retaining means 50 in inner tubular member 20 could be conventional through-holes.

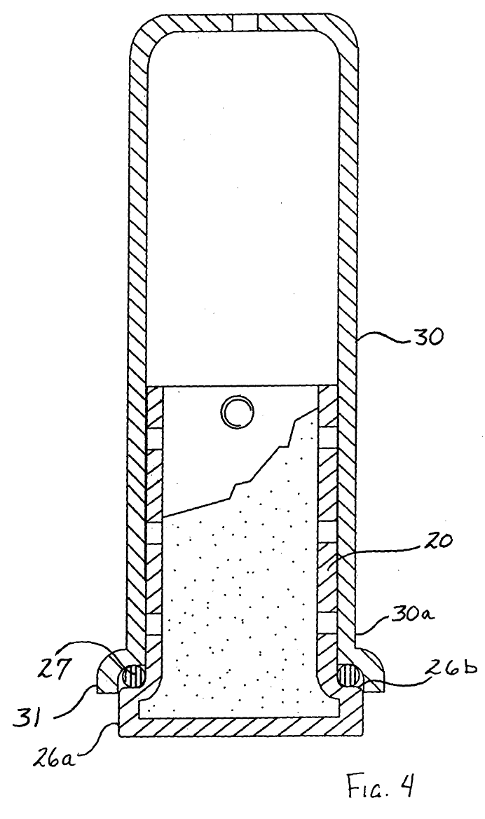

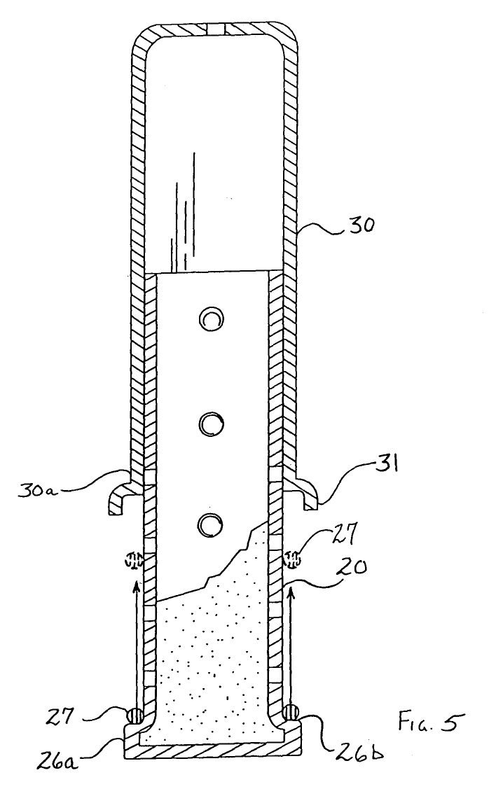

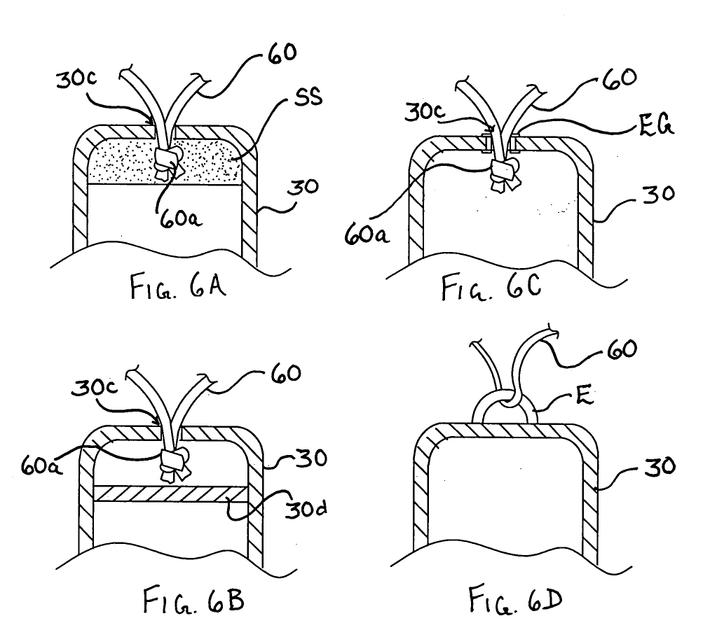

Referring now more specifically to FIGS. 3-4, illustrated therein is an alternate embodiment of scent dispenser 10, wherein the alternate embodiment of FIGS. 3-4 is substantially equivalent in form and function to that of the preferred embodiment detailed and illustrated in FIGS. 1-2 except as hereinafter specifically referenced. Specifically, the embodiment of FIGS. 3-4 incorporates elastomeric O-ring 27 frictionally disposed around base 26a of inner tubular member 20, wherein base 26a provides a lip 26b of sufficient area to support and retain O-ring 27 thereon and thereagainst. O-ring 27 is incorporated for multiple purposes as hereinafter described, including for purposes of creating a spill-proof scent dispenser 10. Flange 31 is formed around base 30a of outer tubular member 30, wherein flange 31 is dimensioned to frictionally engage O-ring 27 on lip 26b of inner tubular member 20 when scent dispenser 10 is in a fully-closed position, thereby preventing spill-age of the scented material and/or fluid upon transport of scent dispenser 10, as best illustrated in FIG. 4. Although elastomeric O-ring 27 is utilized for spill-proofing scent dispenser 10, it is contemplated in another alternate embodiment that other suitable spill-proofing mechanisms could be utilized, such as, for exemplary purposes only, a silicone bead, spill-proofing tape, elastomeric sealing washers, or the like. It should further be recognized that elastomeric O-ring 27 provides the advantage of eliminating undesirable vapor loss from the scented material contained within scent dispenser 10. Furthermore, undesirable vapor loss through throughhole 30c of outer tubular member 30, from which cord 60 extends, is preferably eliminated via the incorporation of suitable barriers and/or sealants positioned proximal to, and/or formed in conjunction with, throughhole 30c, as more fully described below and detailed in FIGS. 6A-6C.

Additionally, and as best illustrated in FIG. 5, elastomeric O-ring 27 is selectively removably positionable, slid, or rolled, along the length of inner tubular member 20, thereby providing an adjustable stop means for frictional engagement with flange 31 of outer tubular member 30; thus maintaining outer tubular member 30 at a user-selected position for selectively controlling scent dispersion from scent dispenser 10. In such a configuration, detent retaining means 50 of the above-described preferred embodiment could be eliminated, as elastomeric O-ring 27 would provide the requisite stop means. Furthermore, it is contemplated that elastomeric O-ring 27 should be of sufficient diameter to provide the requisite frictional engagement between flange 31 and the surface of inner tubular member 20, thereby preventing undesired downward and/or upward displacement of outer tubular member 30 relative to inner tubular member 20. Alternatively, detent retaining means 50 could be utilized in conjunction with O-ring 27 to provide a positive limit stop to prevent disengagement of outer tubular member 30 from dispenser 10 under circumstances wherein the user engages inner tubular member 20 and outer tubular member 30 intermediate such detent retaining means 50.

Referring now more specifically to FIGS. 6A-6C, it is contemplated in still another alternate embodiment that throughhole 30c of outer tubular member 30, from which cord 60 extends, could also be spill-proofed via any suitable means. For example, and as best seen with reference to FIG. 6A, a layer of silicone sealant SS could be applied behind top surface 30b of outer tubular member 30 so as to fully cover or seal knotted end 60a of cord 60, and to further seal throughhole 30c. Alternatively, and as best seen with reference to FIG. 6B, barrier wall 30d could be affixed to the inner walls of outer tubular member 30, or integrally formed therewith, thereby sealing end 60a of cord 60, and throughhole 30c, therebehind. As best seen with reference to FIG. 6C, elastomeric grommets EG could be frictionally positioned within throughhole 30c, or integrally formed therewith, wherein cord 60 would frictionally extend therefrom. Other suitable sealants and/or barriers may include, but are not limited to, elastomeric washers, elastomeric rivets, elastomeric rings, plastic melts, and/or the like. In yet another alternate embodiment, and as best illustrated in FIG. 6D, throughhole 30c could be eliminated entirely, wherein cord 60 could be tied to eyelet E formed on top surface 30b of outer tubular member 30. Alternatively, cord 60 could be integrally formed with or attached to outer tubular member 30 during manufacture of same.

Having thus described preferred embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. A scent dispenser comprising:
an outer tubular member comprising a flanged end portion;
an inner tubular member comprising a base and a plurality of apertures for emitting scent therethrough, said inner tubular member dimensioned to be slidably received and retained within said outer tubular member;
said inner and outer tubular members forming a cavity therebetween when said members are engaged; and,
a selectively removably positionable elastomeric sealing means.

2. The scent dispenser of claim 1 wherein said inner and outer tubular members each comprise a rectangular cross-section.

3. The scent dispenser of claim 1 further comprising a plurality of frictionally engageable, nestable detents for removably retaining said outer tubular member at a user-selected position along the length of said inner tubular member.

4. The scent dispenser of claim 1 further comprising a scented material carried within said cavity.

5. The scent dispenser of claim 4 wherein said scented material is carried by an absorbent material.

6. The scent dispenser of claim 1 further comprising means for suspending said scent dispenser.

7. The scent dispenser of claim 6 wherein said means for suspending said scent dispenser is selected from the group consisting of tethers, straps, cords, ties, and cooperating hook-and-loop fasteners.

8. The scent dispenser of claim 1 further comprising a bright color for enhancing the visibility of said scent dispenser.

9. The scent dispenser of claim 1 further comprising a color which blends into the environment for decreasing the visibility of said scent dispenser.

10. The scent dispenser of claim 1, wherein said sealing means is frictionally disposed around said base of said inner tubular member, said base of said inner tubular member comprising a lip having a sufficient area to support and retain said sealing means thereon and thereagainst, and wherein placement of said scent dispenser in a fully-closed position results in said flanged end portion frictionally engaging said sealing means, thereby creating a spill-proof seal between said flanged end portion of said outer tubular member and said base of said inner tubular member.

11. The scent dispenser of claim 1, wherein selectively removably positioning said sealing means a selected distance away from said base of said inner tubular member along the length of said inner tubular member enables said sealing means to function as an adjustable stop means for frictionally maintaining said flanged end portion of said outer tubular member thereagainst, thereby maintaining said outer tubular member at a user-selected position along said length of said inner tubular portion.

12. The scent dispenser of claim 1, wherein said sealing means is selected from the group consisting of elastomeric sealing rings, silicone bead, tape and elastomeric sealing washers.

13. A scent dispenser comprising:
an outer tubular member comprising a flanged end portion;
an inner tubular member comprising a base and a plurality of apertures for emitting scent therethrough, said inner tubular member dimensioned to be slidably received and retained within said outer tubular member;
said inner and outer tubular members forming a cavity therebetween when said members are engaged; and,
a selectively removably positionable elastomeric sealing ring disposed proximal said base of said inner member for providing a spill-proof seal between said base of said inner member and said flanged end portion of said outer tubular member when said flanged end portion of said outer tubular member is frictionally engaged therewith.

14. The scent dispenser of claim 13 wherein said inner and outer tubular members each comprise a rectangular cross-section.

15. The scent dispenser of claim 13 further comprising a plurality of frictionally engageable, nestable detents for removably retaining said outer tubular member at a user-selected position along the length of said inner tubular member.

16. The scent dispenser of claim 13 further comprising a scented material carried within said cavity.

17. The scent dispenser of claim 16 wherein said scented material is carried by an absorbent material.

18. The scent dispenser of claim 13 further comprising means for suspending said scent dispenser.

19. The scent dispenser of claim 18 wherein said means for suspending said scent dispenser is selected from the group consisting of tethers, straps, cords, ties, and cooperating hook-and-loop fasteners.

20. The scent dispenser of claim 13 further comprising a bright color for enhancing the visibility of said scent dispenser.

21. The scent dispenser of claim 13 further comprising a color which blends into the environment for decreasing the visibility of said scent dispenser.

22. The scent dispenser of claim 13, wherein selectively removably positioning said sealing ring a selected distance away from said base of said inner tubular member along the length of said inner tubular member enables said sealing ring to function as an adjustable stop means for frictionally maintaining said flanged end portion of said outer tubular member thereagainst, thereby maintaining said outer tubular member at a user-selected position along said length of said inner tubular portion.

23. A scent dispenser comprising:
an outer tubular member comprising a flanged end portion;
an inner tubular member comprising a base and a plurality of apertures for emitting scent therethrough, said inner tubular member dimensioned to be slidably received and retained within said outer tubular member;
said inner and outer tubular members forming a cavity therebetween when said members are engaged; and,
a selectively removably positionable elastomeric sealing ring moveable from a first position to at least a second position, wherein said first position provides a spill-proof seal between said base of said inner member and said flanged end portion of said outer tubular member when said flanged end portion of said outer tubular member is frictionally engaged therewith, and wherein said at least a second position enables said sealing ring to function as an adjustable stop means for frictionally maintaining said flanged end portion thereagainst, thereby maintaining said outer tubular member at a user-selected position along the length of said inner tubular portion.

24. The scent dispenser of claim 23 wherein said inner and outer tubular members each comprise a rectangular cross-section.

25. The scent dispenser of claim 23 further comprising a plurality of frictionally engageable, nestable detents for removably retaining said outer tubular member at a user-selected position along the length of said inner tubular member.

26. The scent dispenser of claim 23 further comprising a scented material carried within said cavity.

27. The scent dispenser of claim 26 wherein said scented material is carried by an absorbent material.

28. The scent dispenser of claim 23 further comprising means for suspending said scent dispenser.

29. The scent dispenser of claim 28 wherein said means for suspending said scent dispenser is selected from the group consisting of tethers, straps, cords, ties, and cooperating hook-and-loop fasteners.

30. The scent dispenser of claim 23 further comprising a bright color for enhancing the visibility of said scent dispenser.

31. The scent dispenser of claim 23 further comprising a color which blends into the environment for decreasing the visibility of said scent dispenser.
